(19) United States
(12) Patent Application Publication
(10) Pub. No.: US 2007/0167690 A1
(43) Pub. Date: Jul. 19, 2007

(54) Mind-body correlation data evaluation apparatus and method of evaluating mind-body correlation data

(75) Inventors: Masako Miyazaki, Edmonton (CA); Tatsuaki Ataka, Nagareyama-shi (JP); Miho Kameyama, Tokyo (JP); Takayoshi Togino, Tokyo (JP)

Correspondence Address: FRISHAUF, HOLTZ, GOODMAN & CHICK, PC, 220 Fifth Avenue, 16TH Floor, NEW YORK, NY, 10001-7708, US

(73) Assignees: OLEMI Trading Inc., Edmonton (CA); Olympus Corporation, Tokyo (JP)

(21) Appl. No.: 11/637,830

(22) Filed: Dec. 12, 2006

(30) Foreign Application Priority Data
Dec. 19, 2005 (JP) JP 2005-364728

(51) Int. Cl. A61B 5/00

(52) U.S. Cl. 600/300

(57) ABSTRACT

A mind-body correlation data evaluation apparatus includes a stimulation providing section which provides stimulation to motivate an action of a subject, a physiological response data collecting section which collects physiological response data of the subject, a subjective rating and action rating data collecting section which collects subjective rating data and action rating data of the subject, a control and calculating section which evaluates a mind-body state of the subject based on the stimulation, and at least one of the physiological response data, the subjective rating data, and the action rating data, and a data feed-back section which feeds back to the subject at least the mind-body state of the subject.

### CROSS-REFERENCE TO RELATED APPLICATION

The present application is based upon and claims the benefit of priority from the prior Japanese Patent Application No. 2005-364728 filed on Dec. 19, 2005; the entire contents of which are incorporated herein by reference.

### BACKGROUND OF THE INVENTION

1. Field of the Invention

The present invention relates to a mind-body correlation data evaluation apparatus which collects mind-body correlation data of a subject individual, and evaluates the mind-body correlation data, and a method of evaluating mind-body correlation data.

2. Description of the Related Art

A data collecting apparatus which measures and records a response of a human being, such as a physiological response, subjectivity and an action of a subject, to a sound, a temperature, and a brightness etc., has hitherto been disclosed in Japanese Patent No. 2772413. This data collecting apparatus collects environmental information of a surrounding of the subject, physiological data, subjective rating data, action rating data, and environmental information data of the subject in that environment.

However, in the apparatus using the conventional technology, the environment around the subject is not to be changed actively. Therefore, the physiological response, the subjectivity, and the action etc. of the subject cannot be measured and recorded when the environment of the surrounding is changed. Moreover, so far, a primary user of the measured data has been a person into architecture etc. who provides the environment. Therefore, a general public who has provided the data who becomes a secondary user, feeding back of the data to the subject has not been considered much.

### SUMMARY OF THE INVENTION

The present invention is made in view of the abovementioned circumstances, and an object of the present invention is to provide a mind-body correlation data evaluation apparatus which supports changing of a mind-body state by a subject, by imparting various stimulations to the subject, then measuring and recording a physiological response, subjectivity, and an action of the subject, and evaluating and feeding back the response, and a method of evaluating mind-body correlation data.

According to the present invention, it is possible to provide a mind-body correlation data evaluation apparatus which includes a stimulation providing section which provides stimulation to motivate an action of a subject, a physiological response data collecting section which collects physiological response data of the subject, a subjective rating data collecting section which collects subjective rating data of the subject, an action rating data collecting section which collects action rating data of the subject, an evaluating and calculating section which evaluates a mind-body state of the subject based on the stimulation and at least one of the physiological response data, the subjective rating data, and the action rating data, and a feed back section which feeds back to the subject at least the mind-body state of the subject.

### BRIEF DESCRIPTION OF THE DRAWINGS

FIG. 1 is a diagram showing functional blocks of a mind-body correlation data evaluation apparatus according to a first embodiment;

FIG. 2 is a diagram showing an internal structure of the mind-body correlation data evaluation apparatus according to the first embodiment;

FIG. 3 is a diagram showing an external structure of the mind-body correlation data evaluation apparatus according to the first embodiment;

FIG. 4 is a diagram showing a schematic structure of a chamber environment operating section of the first embodiment;

FIG. 5 is a diagram showing a schematic structure of a face-photo taking section of the first embodiment.

FIG. 6 is a flowchart showing a schematic procedure of a method of evaluating mind-body correlation data in the first embodiment;

FIG. 7 is a flowchart showing a detail procedure of the method of evaluating mind-body correlation data in the first embodiment;

FIG. 8A and FIG. 8B are diagrams showing examples of stimulation;

FIG. 9A and FIG. 9B are diagrams showing examples of subjective rating data;

FIG. 10A and FIG. 10B are diagrams showing examples of combinations of stimulations;

FIG. 11 is a diagram describing a method for selecting a stimulation for a subject;

FIG. 12 is a diagram showing a change in emotions of a subject due to stimulation;

FIG. 13 is a diagram showing examples of combinations of stimulations matched with evaluation data;

FIG. 14 is a diagram showing a schematic structure of a mind-body correlation data evaluation apparatus according to a second embodiment;

FIG. 15 is a flowchart showing a procedure for a method of evaluating mind-body correlation data according to the second embodiment;

FIG. 16 is a diagram showing an internal structure of a mind-body correlation data evaluation apparatus according to a third embodiment; and

FIG. 17 is a diagram showing an external structure of the mind-body correlation data evaluation apparatus according to the third embodiment.

### DETAILED DESCRIPTION OF THE INVENTION

Embodiments of a mind-body correlation data evaluation apparatus and a method of evaluating mind-body correlation data according to the present invention will be described below in detail with reference to accompanying diagrams. However, the present invention is not restricted by these embodiments.

### FIRST EMBODIMENT

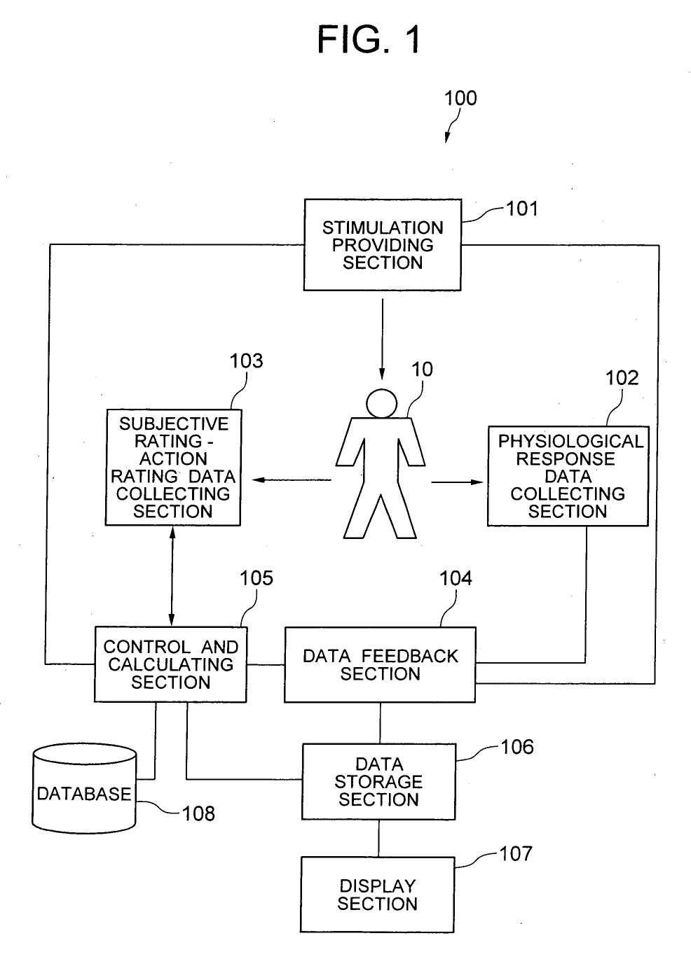

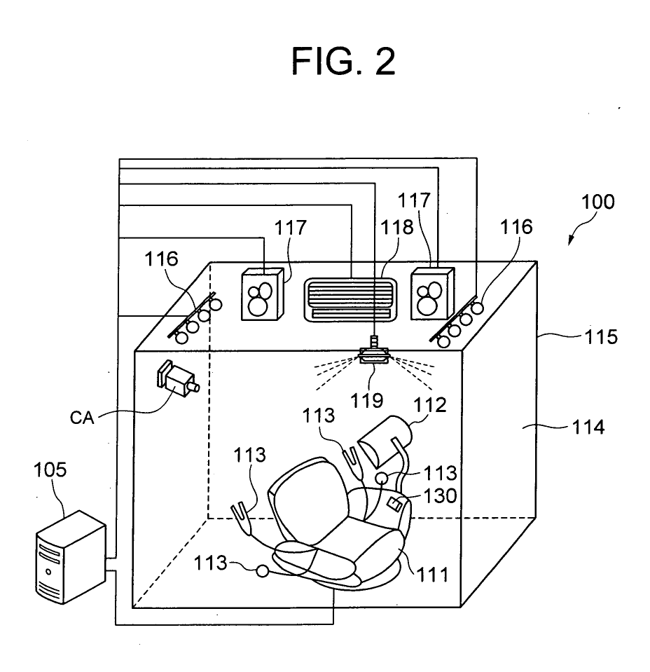

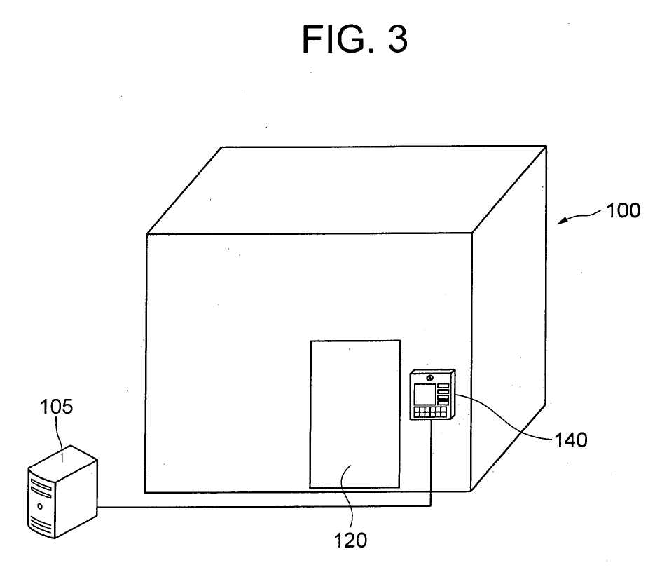

FIG. 1 shows functional blocks of a mind-body correlation data evaluation apparatus 100 according to the present invention. FIG. 2 shows a schematic structure of the mind-body correlation data evaluation apparatus 100. FIG. 3 shows an external structure of the mind-body correlation data evaluation apparatus 100.

As shown in FIG. 1, the mind-body correlation data evaluation apparatus 100 includes a stimulation providing section 101, a physiological response data collecting section 102, a subjective rating-action rating data collecting section 103, a data feed-back section 104, a control and calculating section 105, a data storage section 106, a display section 107, and a database 108.

Concretely, as shown in FIG. 2, the mind-body correlation data evaluation apparatus 100 includes a chair 111 which is provided in a housing 115 which is a rectangular parallelepiped. A subject 10 (not shown in the diagram), opens a door 120 and enters the housing 115. Then, the subject 10 sits in the chair 111. An illuminating section 116, a speaker 117, an air-conditioning section 118, and an image providing section 119 are provided on a ceiling of the housing 115.

The illuminating section 116 includes an illuminating unit which is capable of emitting light by changing a color temperature, a color, and a brightness of illumination. The speaker 117 supplies music and audio etc. The air-conditioning section 118 controls a temperature and humidity. Moreover, the air-conditioning section 118 has an aroma (smell) generating function of supplying smells of various types.

The image providing section 119 is a recording and reproducing unit or a projector which is provided with a function of projecting an image from a video tape and an optical disc, and a function of reproducing an audio. A wall 114 on an inner side of the housing 115 is provided with a function of a screen. The wall 114 may be a structure in which concavo-convex structure of a wall surface are changeable according to the requirement, and a structure in which a form of a screen which encompasses the subject 10 is changeable. Moreover, a method of providing visual stimulation can be realized by using a visual stimulation providing apparatus in the form of a goggle, apart from a projector.

A camera input section 112 is provided in front of the subject 10. The camera input section 112 records actions such as movements and expressions of a face of the subject 10. For example, a photograph of the subject 10 immediately after entering the mind-body correlation data evaluation apparatus 100 and immediately before leaving the mind-body correlation data evaluation apparatus 100 is taken by the camera input section 112, and recorded.

Moreover, a key board (not shown in the diagram) is provided in the camera input section 112. The subject 10 can create a diary and a text etc. related to emotions by using the key board. The subject 10 can also select and determined icons which express feelings. Furthermore, an audio of the subject 10 can be acquired by using a microphone (not shown in the diagram). Thus, the subject 10 is asked to specify state of mind, and feelings by entering characters, or a simple method.

Moreover, a plurality of electrodes 113 is provided to the chair 111. The subject 10 puts the electrodes 113 on a body. Physiological response data of the subject 10, such as an electroencephalogram, an electrocardiogram, a pulse wave, an electrodermal activity can be detected non-invasively by the electrodes 113. Moreover, since the electrodes 113 can be structured to be small, portion restraining the subject 10 may be small. Furthermore, it is also possible to put electrodes of an electromyography on mimetic muscles (muscles of facial expressions) of the subject 10, and to speculate a change in the mimetic muscles of the subject 10.

Moreover, it is also possible to provide in the housing 115 a camera CA which takes images of actions, behavior, and postures of the subject 10. Accordingly, it is possible to acquire action rating data of the subject 10 for a stimulation which is imparted.

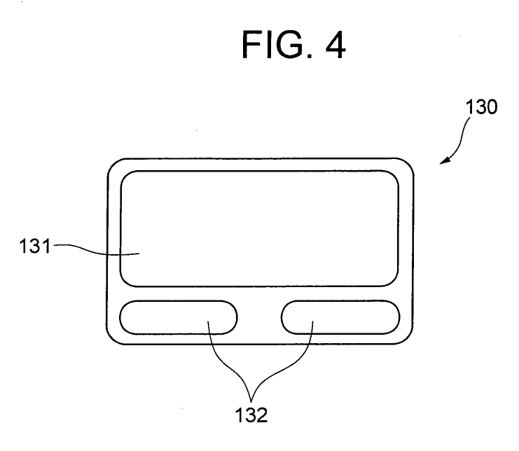

FIG. 4 shows a schematic structure of a chamber environment operating section 130 which is provided in the housing 115. The chamber environment operating section 130 includes a touch-panel operating section 131 and an operating instruction narration transmitting section 132. The operating instruction narration transmitting section 132 conveys information for setting and changing a chamber environment, to the subject 10 by an audio guide. Moreover, the subject 10 can set the chamber environment such as the temperature and/or humidity etc. mentioned above to a desired condition, by the touch-panel operating section 131.

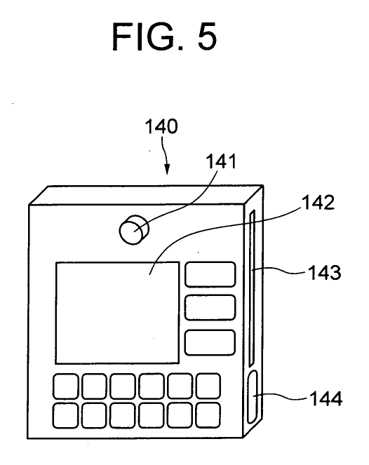

FIG. 5 shows a schematic structure of a face photo taking section 140. The face photo taking section 140 is provided near the door 120 for entry and exit to and from the chamber as shown in FIG. 3. Moreover, the face photo taking section 140 includes a camera 141, a display 142, a data sheet discharging section 143, and a USB connecting section 144.

The camera 141 takes a photo of a face of the subject 10 before entering into and after leaving the mind-body correlation data evaluating apparatus 100. The display 142 displays expressions of the face photographed by the camera 141. Moreover, the data sheet discharging section 143 discharges a sheet on which data related to the evaluation is recorded, after the evaluation of the mind-body correlation data by a procedure which will be described later is over. Furthermore, the subject 10 can also store data related to the evaluation in a recording medium such as a portable memory, via the USB connecting section 144.

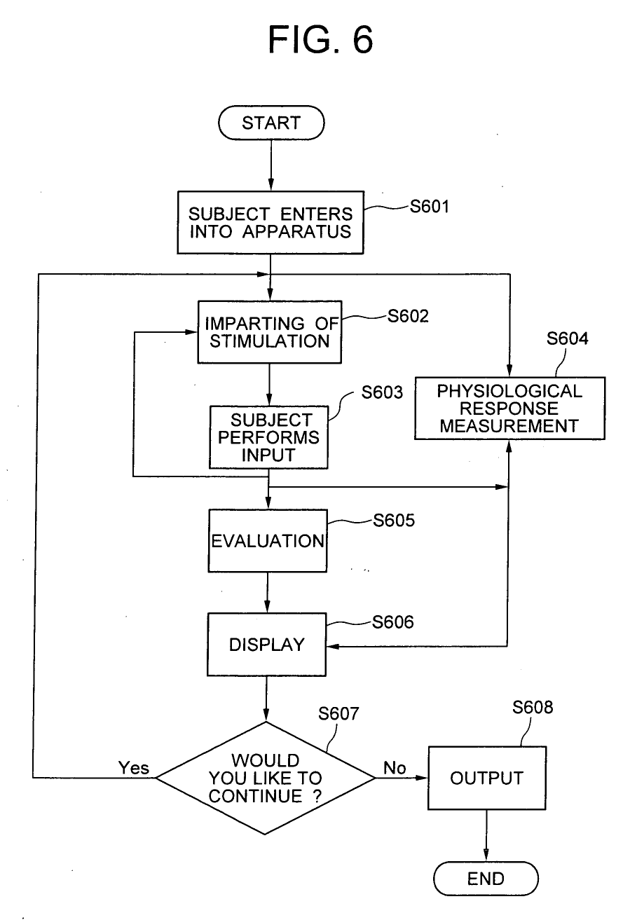

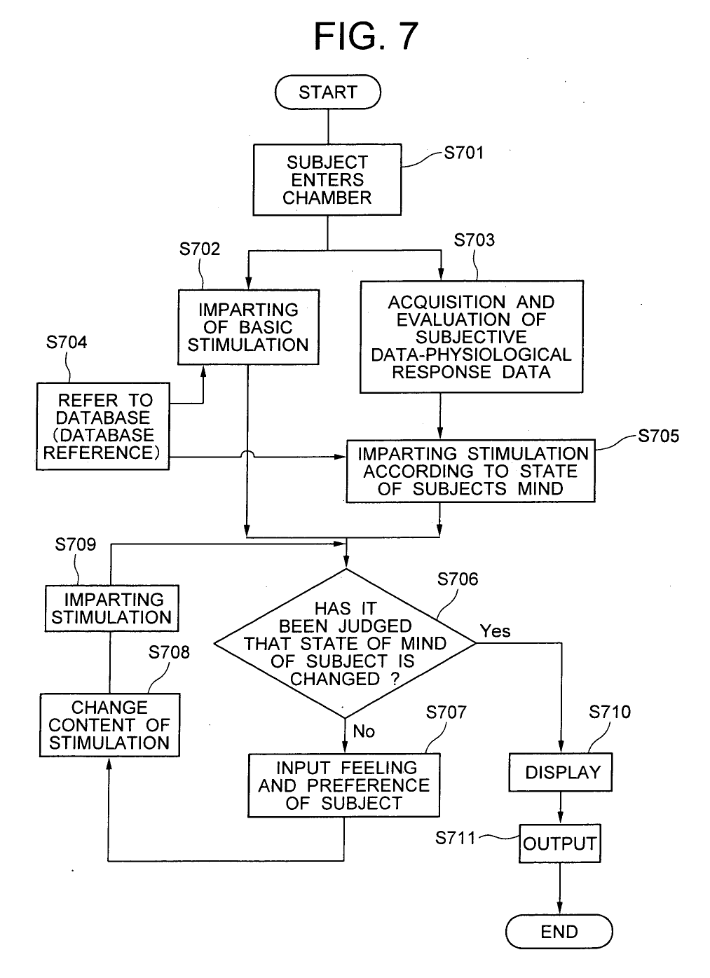

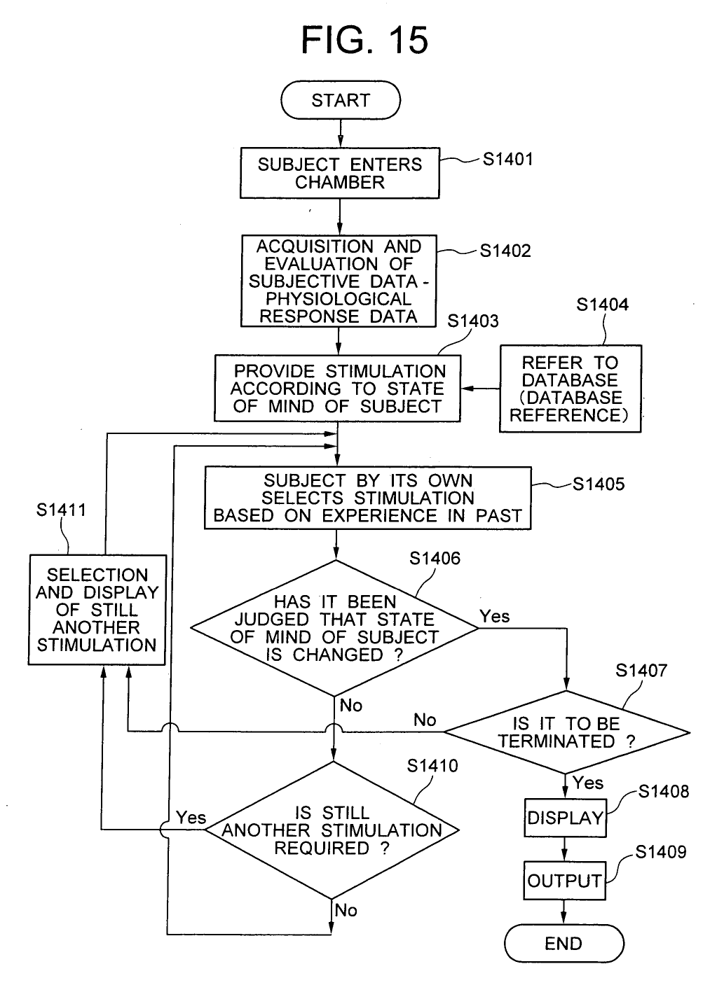

Next an evaluation procedure of a mind-body state of the subject 10 will be described below. FIG. 6 is a flowchart showing an evaluation procedure of a simplified basic concept. Moreover, in an actual evaluation procedure, various operations are performed by making various combinations of stimulation, evaluation (judgment), and input etc. For example, FIG. 7 is a flowchart showing one example of the actual evaluation procedure. FIG. 15, which will be described later, is another example of the actual evaluation procedure. First of all, the evaluation procedure will be described based on FIG. 6.

Normally, an individual preference and feelings vary for each subject. Therefore, when stimulation with the same conditions is provided to the subject, a response to the stimulation varies for each individual. In the first embodiment, a sort of a model mode is performed in which, a judgment of, upon receiving which sort of stimulation, a change in the mind-body state of one's own, particularly a change to a stable pleasant state can be experienced is made.

Apart from the basic preference etc. of the subject 10 such as a favorite color and a favorite music, types and a magnitude of a stimulation which is desired are considered to vary according to the mind-body state of the subject 10. For example, in case of a color, even if the subject likes “blue color” in daily life, some times a stimulation associated with “red color” may be considered to be pleasant for the mind-body state at that time. Thus, it is possible to relate the mind-body state of the subject 10, with the stimulation.

To start with, at step S601, the subject 10 enters the mind-body correlation data evaluation apparatus 100 through the door 120. At this time, as it has been mentioned above, a face photo of the subject 10 is taken by the camera 141. Moreover, in the model mode, the mind-body correlation data evaluation apparatus 100 does not have past evaluation data related to the subject 10.

At step S602, the stimulation providing section 101 imparts stimulation to the subject 10. At step S603, the subject 10 inputs in the mind-body correlation data evaluation apparatus 100, subjective rating data such as a feeling and a sentiment upon receiving the stimulation, by creating a diary and audio data.

Moreover, at step S604, the physiological response data collecting section 102 collects physiological response data of the subject 10. The physiological data collecting section 102 is a section which measures and processes non-invasively and unrestrictedly the physiological response from the subject 10. For example the physiological response data collecting section 102 measures a physiological response such as the electrocardiogram, the pulse wave, and the electroencephalogram from the subject 10. Further, the physiological response data collecting section 102 extracts a physiological index from the physiological response which is measured, and outputs this physiological index.

At step S605, the control and calculating section 105 evaluates the mind-body state of the subject 10 based on the stimulation, physiological response data, the subjective rating data, and the action rating data which are imparted to the subject 10. Hereafter, at least one of the physiological response data, the subjective rating data, and the action rating data in a relationship of the stimulation imparted with the response of the subject 10 will be called as “mind-body correlation data” where deemed appropriate.

At step S606, the display section 107 displays an evaluation result, a type of the stimulation, and a state of an amount of stimulation. At step S607, a judgment of whether or not the evaluation is to be performed continuously is made. When a judgment result at step S607 is true (Yes), the process is moved to step S602. When the judgment result at step S607 is false (No), at step S608, the evaluation result, the amount of stimulation etc. are output.

Steps S606 and S608 correspond to a step of feed back. For example, at step S608, it is possible to process the data of the subject 10 which is integrated, such that the data is easily understandable by the subject, and to print and output the data on a paper, as well as to record electronic data in a recording medium only by that person (the subject 10). Indicating the data processing, for example, for the physiological response, by one axis of whether a sympathetic nervous system is predominant or a parasympathetic nervous system is predominant, can be considered. The subjective rating data and the action rating data can communicate objectively a correlation of mind and body to the subject, by displaying a result of the physiological response data together with a type or an amount of the stimulation.

The evaluation procedure in further detail will be described by using FIG. 7. At step S701, the subject 10 enters the mind-body correlation data evaluation apparatus 100. Next, the control and calculating section 105 performs one of the step S702 and step S703. A selection of the execution of the step S702 and step S703 may be performed by the subject 10, or as to which step is to be executed may be registered in advance in the control and calculating section 105.

First of all, step S702 will be described below. At step S702, the control and calculating section 105 selects a stimulation and an amount of stimulation (hereinafter called as “stimulation amount”) having a standard content, and imparts to the subject 10. Information related to the stimulation is stored in the database 108.

At step S704, the control and calculating section 105, at the time of selecting the stimulation of the standard content, refers to the database 108. Information such as information in the following table 1 is stored in the database 108. Further, for each stimulation, a stimulation which is provided is displayed. Moreover, individual data of the subject 10 can also be stored in the database 108. The individual data includes attributes of the subject such as of age, sex, mother language, and information of environment in which the subject 10 was brought up. The subject 10 can perform a management of the individual data by using a storage medium other than the mind-body correlation data evaluation apparatus 100. At this time, the mind-body correlation data evaluation apparatus 100 reads the individual data of the subject 10 from the storage medium, and stores in the database 108. Moreover, a protection and management of the information stored in the database 108 may be performed by the subject 10, or by a management organization requested by the subject 10.

Here, it is possible to impart an aroma (smell) corresponding to nostalgic photographs of the past, and various scenes (visual scenes). In table 1, “ON” indicates that a state in which the stimulation is imparted, “OFF” indicates that a state in which the stimulation is not imparted, and “CHANGE” indicates that a state in which the amount of simulation can be increased or decreased can be selected. The indications are same for all the tables.

TABLE 1

| | | | | |
|---|---|---|---|---|
| (1) | Sound | ON | OFF | CHANGE |
| (2) | Smell | ON | OFF | CHANGE |
| (3) | Color | ON | OFF | CHANGE |
| (4) | Light | ON | OFF | CHANGE |
| (5) | Air conditioning | ON | OFF | CHANGE |
| (6) | Work | ON | OFF | CHANGE |
| (7) | Image | ON | OFF | CHANGE |

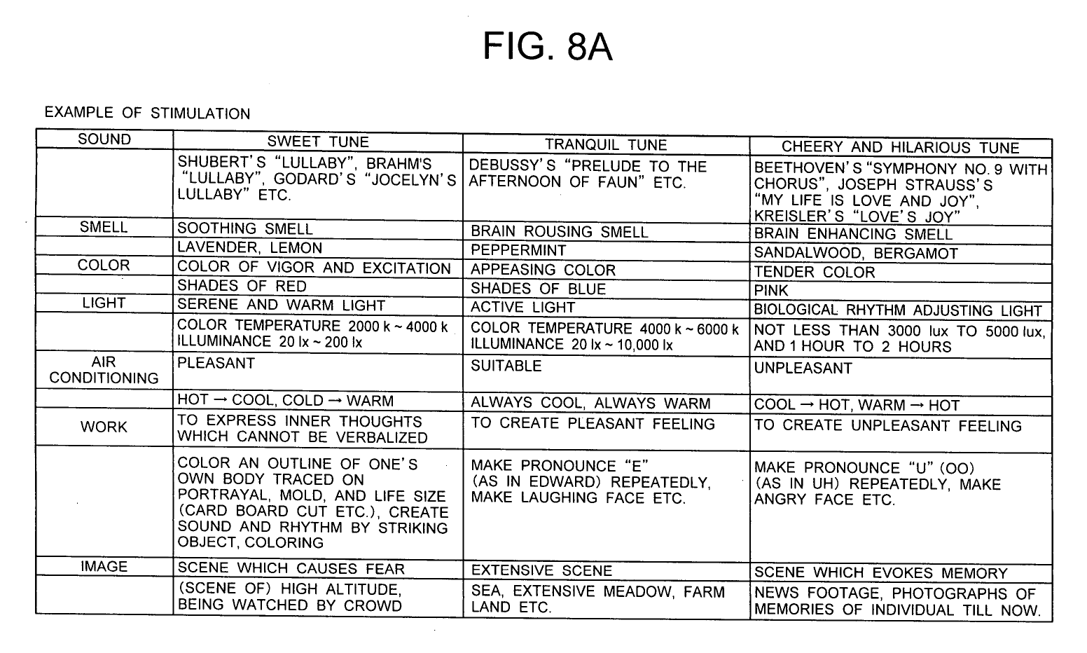

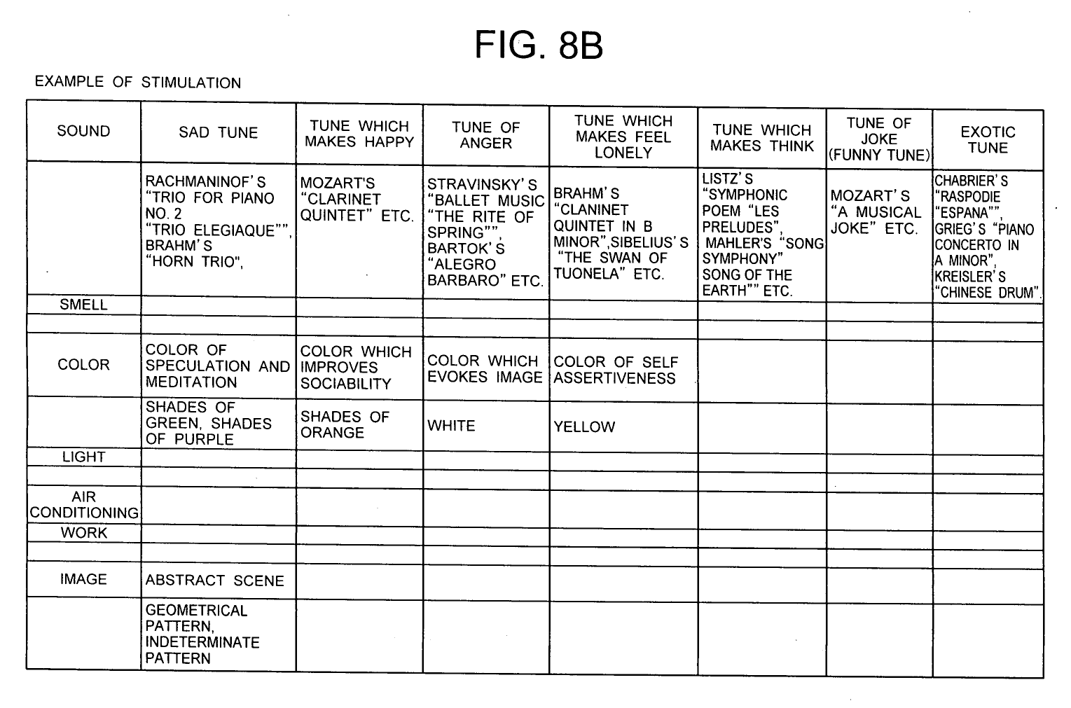

FIG. 8A and FIG. 8B show examples of content of stimulation in further details. Sound, smell, color, light, air conditioning, work, and image are given as examples of stimulation. For example, description will be made with reference to the sound as a representative example. There are various types of sounds, particularly music, namely a “sweet tune”, a “tranquil tune”, a “cheery and hilarious tune”, a “sad tune”, a “tune which makes happy”, a “tune of anger”, a “tune which makes feel lonely”, a “tune which makes think”, a “tune of joke, and an “exotic tune”.

Next, the stimulation provided by the stimulation providing section 101 will be described in further detail. For example, “an example of stimulation which expresses mind” and “an example of stimulation which affects mind” will be described below.

(Example of Stimulation which Expresses Mind)

Art Therapy:

Regarding an art, emotions and opinion of self-expression have been known in the first place. Moreover, expressing these emotions and opinions turns into an art. Whereas, in a case of art therapy, the emotions and opinions of self need not be known. In the art therapy, realizing the emotions and opinion of self in a process of a creative activity is aspired.

In the mind-body correlation data evaluation apparatus 100 of the first embodiment, tools required for a virtual creativity, instead of canvass and coloring matter are provided. Moreover, it is possible to makes a structure in which, when the subject 10 touches a wall, a color is changed, or a structure in which, the color of the wall changes by a body temperature of the subject 40. Accordingly, the subject can express while having fun, emotions and opinions which the subject is not conscious of.

Projective Method:

When a human being sees an object which does not have a definite shape, there is a tendency to see the object under an influence of a state in the mind. Such a phenomenon of mind is called as projection. “Projective method” is a psychological test in which the projection which is a phenomenon of mind is used. For example, Rorschach test is a known psychological test.

In the Rorshach test, several plates having stains of ink are shown to the subject 10, and the subject 10 is asked to reply freely according to the observation of the subject 10. From a tendency of replying, an attempt is made to figure out a way of thinking and an emotional state of the subject 10.

In the mind-body correlation data apparatus 100, plates are prepared. These plates are shown to the subject 10. Next, the subject 10 is asked how the plate is viewed. Answer data is collected, and the tendency of answering is made clear. Next, the tendency is fed back to the subject 10.

Apart from this, the plate may be turned to be a picture which shows a vague situation. This picture may be shown to the subject 10, and a nature of the subject 10 may be analyzed according to a content of a story imagined from the picture (thematic apperception test). The subject 10 may be asked to draw a tree, and the nature of the subject 10 may be diagnosed from the picture (Baum test).

Next, a psychological test will be described.

Performance Test:

In a performance test, the subject 10 is made to perform a certain work such as addition, and from an approach attitude and a result of the work, the nature of the subject 10 is judged. Uchida-Kraepelin Performance Test is a typical example of the performance test. A tendency of the subject 10 is understood from a work curve in which an amount of work per minute is plotted.

For example, there is a pattern in which a lot of work is done in the beginning, and a short time later, the amount of work is declined due to getting tired or bored. Conversely, there is a pattern in which the amount of work is less in the beginning, and goes on increasing gradually. In the mind-body correlation data evaluation apparatus 100, the Uchida-Kraepelin Performance Test can be adopted. A work is provided to the subject 10, and a quality and the amount of work per minute is recorded. Accordingly, it is possible to make clear the pattern of the subject 10, and to feed back to the subject 10.

Next, “an example of stimulation which affects mind” will be described below.

Providing Aroma:

A preference for aroma has been known to be influenced by an education, psychological factors, and a gender difference (for example, refer to page 43 of Human Environmental Studies in Journal of Architectural Institute of Japan, published in 1998 by Asakura Publishing Co., Ltd., Tokyo). Based on. this, it is considered that by finding a preference of smell and recording the preference of smell, an index which reflects a personality and a daily psychological state of an individual can be obtained.

A smell is known to have a Proust effect in which the memory is recalled by a smell. When various smells are discharged from the stimulation providing section 101, an effect in which memory which has been forgotten by the subject 10 is brought back can be anticipated.

Moreover, aroma is released from essential oils which are extracted from flowers, seeds, fruits, and leaves. Such aroma is known to have a positive effect on mind. Such effect is also confirmed by research.

For example, a research in which it was found that various smells have an effect on a mental fatigue of healthy adult man and woman (refer to pages 100 to 108 (2000) of report on achievements for the year 1999 of a research related to a method of recovery from fatigue for fitness, and research on the actual situation of fatigue, Welfare Science Research Grant—Health Science multidisciplinary research project, study by effects of smell, measure for evaluation, and psychophysiological method for uneasiness and fatigue by a mental work load, by Teruo Kitani, Shunkichi Endo, Hiroko Suzuki et. al.). According to this literature, smell of peppermint, bergamot, lavender, and sandalwood have an effect of reducing uneasiness and subjective fatigue degree. Particularly, it has been revealed that bergamot and lavender which have a calming effect are effective in reducing the uneasiness.

Peppermint has an effect of increasing significantly a fast component of brain wave (electroencephalogram), and a degree of alertness. Bergamot and sandalwood have a positive effect on selective attention power. It has been indicated that lavender has a positive effect on information processing of brain.

When such an aroma is released from the stimulation providing apparatus 101, effects such as the recovery from fatigue, and an improvement in the alertness, are brought about.

Providing Music:

The subject 10 selects a music which is thought to have the maximum calming effect by the subject 10, and listens to the selected music for 30 minutes. Subjectively, a reduction in a degree of nervousness, uneasiness, fatigue, and irritation, and en enhancement in a degree of happiness, and relaxation are observed. Moreover, by an objective index, a predominance of the parasympathetic nervous system is observed from an analysis of R-R interval of the electrocardiogram. Thus, the music which is selected upon judging by oneself to have the calming effect is known to have the calming effect (refer to proceedings of Fifth Japanese Music Therapy Association Academic Meet, 2005, Study of search for objective and scientific index used in a judgment of effect of the music therapy by Mayu Kondo, Munetaka Haida, Masaru Murakami, Shun-ichiro Izumi, Tsunehisa Makino, Shigeki Okino, and Tetsuo Shimizu. Therefore, a music which one feels to have a calming effect is selected, and the selected music is played by the stimulation providing section 101. Accordingly, the calming effect is anticipated for the subject who has selected the music.

Providing Color:

Colors are known to have a function of determining a physiologic state of a human being. For example, light of a color such as red and yellow have functions of stimulating an autonomous nervous system, raising a blood pressure, and increasing a respiration rate and blinking of eyes. Whereas, a light of a cold color such as blue and green, does not stimulate the autonomous nervous system, but have a function of reducing the respiration rate (refer to Liberman, J.: Light, Bear & Company Inc., 1991 (pages 40 to 42 of “Medical Science of Light and Mechanism of Healing conduced by Light and Colors”, translated by Daisuke Iimura, published by Nihon Kyobunsha (Publishing) Co., Ltd., 1996)).

When a warm color and a cold color are provided according to the state of the subject 10, it is possible to excite the subject 10 physiologically, or to bring him to a resting state.

Moreover, there has been a progress in a psychological and clinical research of a psychological message of a color. According to this research, it has been revealed that even while there is a common perception for a certain color having a psychological image, in detail, the perception vary according to a person (refer to pages 110 to 147 of Color Psychology which explains well the General Knowledge of Illustration by Keiko Yamawaki, published by Natsume Publishing Co., Ltd. in 2005).

This is because in an image of a color the following three components (1), (2), and (3) are tangled in a complicated manner.
(1) Information obtained in a process of evolution of the human being since the primitive age
(2) Information which has been taken over according to countries and cultures, and
(3) Information which has emerged from experiences of individuals.

Consequently, when a judgment is made based only on a color selected by the subject 10, it is possible to presume a normal psychological state of the subject 10. More preferably, it is desirable to acquire and store in advance an image (impression etc.) of main colors which the subject 10 has. Further, based on the color selected by the subject 10, an image which is stored according to the subject 10 is applied. Accordingly, it is possible to speculate the psychological state of the subject 10.

Moreover, relevance of a color preference and character has been known. An example of Birren color analyst will be given. This is a relevance of the color preference and character based on a case study (refer to pages 204 to 217 of “Unraveling A Mystery of Birren Science of Color” written by Faber Birren, translated by Kunio Sato (“Color and Human Response), published by SEIGA BOOK (Publishing Co., Ltd.) in 2003). According to this, a person who prefers red color is full of interest in diplomatic intentions. Males and females who prefer red color have rich impulsiveness, excellent sports ability, and are sexy. Moreover, they are a sort of people who say whatever comes to their mind irrespective of whether it is true or not. Apart from red color, such a relevance of the color preference and character has been known for pink, orange, yellow, greenish yellow, green, bluish green, shades of purples, brown, white, black, and gray colors. When the color selected by the subject 10 and this information are corresponded, in the control and calculating section 105, the stimulation can imparted upon having considered the nature of the subject 10, and it is helpful in creating options for the stimulation to be imparted.

Providing Light:

A dark environment without light has a function of calming a motor nerve, and makes a person feel relaxed. Moreover, in the dark environment with poor light, an outline of an object becomes obscure. A border of an object is seen only blurred, and a sense of distance from an object becomes ambiguous. Religious buildings such as a church normally have a dark space inside. This is because it is considered that in a dark place, human being tends to focus the attention to inner thoughts (page 24 of Human Environmental Studies in Journal of Architectural Institute of Japan, published in 1998 by Asakura Publishing Co., Ltd.). Based on this, when an amount of light from the stimulation providing section 101 is decreased, effects such as calming the subject 10 and making the subject 10 to focus attention to inner thoughts are anticipated.

Next, a behavior therapy support will be described below.

Assertive Training and Modeling:

Some people, due to a behavior pattern of restraining without saying what they want to say, result in having stress. An assertive training is one of the trainings for such people to not to have stress. The assertive training is a training of learning to express by words, attitude, and behavior, what one wants to convey, so that it is acceptable to other person.

For example, when the subject 10 selects what the subject 10 wants to assert, from a menu which is prepared in advance, a scene in which the assertion is used is displayed by a four-frame cartoon. First time, it is memorized by looking at a speech and an action of the scene of assertion of a hero. Second time, the subject acts by his own, a role of the hero while imitating a sample. Accordingly, it is possible to make a training such that the subject 10 can assert at a scene where the subject could not assert own opinion so far.

Use of Systematic Desensitization Method:

This is a method in which, with a fear and an uneasiness as a main object, an intention is to make not to feel the fear and uneasiness (desensitization effect) in stages (systematically). By arranging this, for example for the subject 10 who gets nervous upon appearing in front of people, first of all, in the mind-body correlation data evaluation apparatus 100, the aroma, light, and sound are adjusted such that the subject 10 can relax. Next, when the subject 10 is relaxed, in the relaxed state, a scene of feeling the nervousness is played by a footage (video) and sound with a raised feeling of presence. By linking such stimulation with a reaction of relaxation, the subject 10 is let to have a feeling in the body of being relaxed from the nervousness. By repeating this, and by providing scenes leading to increased nervousness according to circumstances, a training linked with the reaction of relaxation is carried out. Finally, the procedure is repeated till the subject 10 stops feeling the nervousness at an actual scene.

Next, step S703 will be described below. At step S703, the physiological response data collecting section 102 and the subjective rating-action rating data collecting section 103 collect the physiological response data, the subjective rating data, and the action rating data. An example of the physiological response data is given in table 2.

TABLE 2

| | | | | |
|---|---|---|---|---|
| (1) | Electroencephalogram | ON | OFF | CHANGE |
| (2) | Pulse wave | ON | OFF | CHANGE |
| (3) | Electrodermal activity | ON | OFF | CHANGE |
| (4) | Body temperature | ON | OFF | CHANGE |
| (5) | Pupil diameter | ON | OFF | CHANGE |
| (6) | Electrocardiogram | ON | OFF | CHANGE |
| (7) | Oxygen saturation | ON | OFF | CHANGE |

Moreover, an example of the action rating data is given in table 3.

TABLE 3

| | |
|---|---|
| (1) | Draw a picture |
| (2) | Do coloring |
| (3) | Mold a form out of clay |
| (4) | Trace own body and color in |
| (5) | Make a sound |
| (6) | Pronounce “E” (as in Edward) repeatedly |
| (7) | Make a laughing face |
| (8) | Pronounce “OO” (as in uh) repeatedly |
| (9) | Make an “angry face” |

Moreover, regarding the subjective rating data, for example, a display of a feeling which is not expressed in the subject's speech, a judgment is made based on data given in table 4 for example.

TABLE 4

| | | | | |
|---|---|---|---|---|
| (1) | Expression | ON | OFF | CHANGE |
| (2) | Audio (voice) | ON | OFF | CHANGE |
| (3) | Posture | ON | OFF | CHANGE |
| (4) | Action which appear repeatedly | ON | OFF | CHANGE |
| (5) | Spatial action | ON | OFF | CHANGE |

The subjective rating data can be acquired by a method given in table 5

TABLE 5

| | |
|---|---|
| (1) | Measurement of a posture, a gesture, and an action by a video |
| | camera |
| (2) | Shooting of expressions by a camera |
| (3) | Information recording, and subjectivity input by an audio |
| | recording section |
| (4) | Recording of nonverbal communication by a grip strength |
| | sensor |
| (5) | Entry of events in the form of a diary |
| (6) | Recording of emotions by a face scale |
| (7) | Recording of emotions by a free entry |

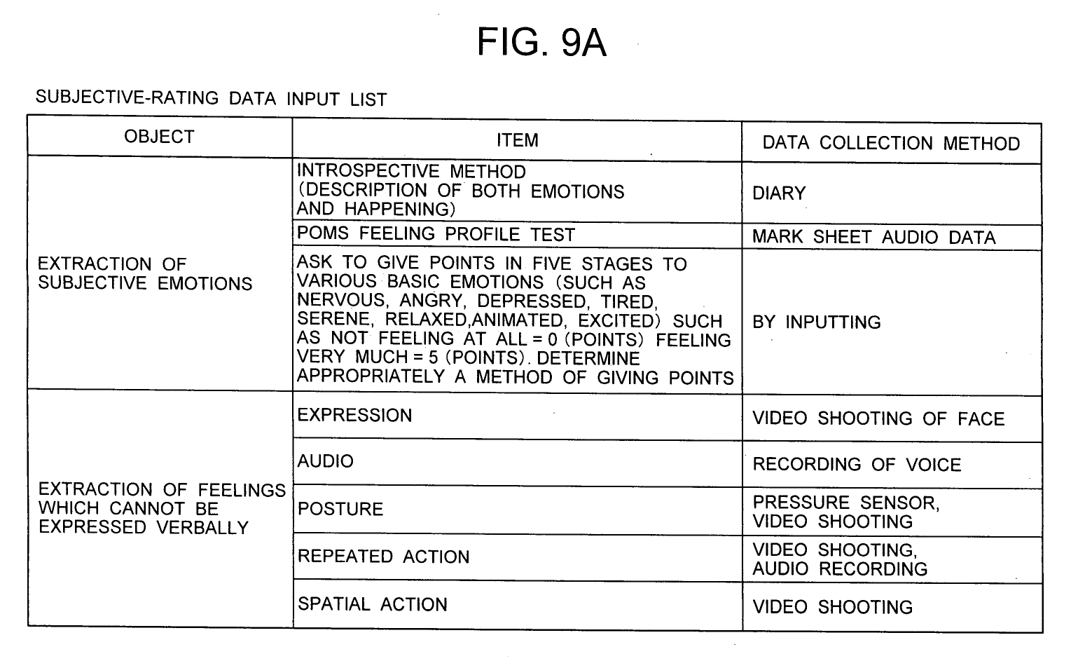

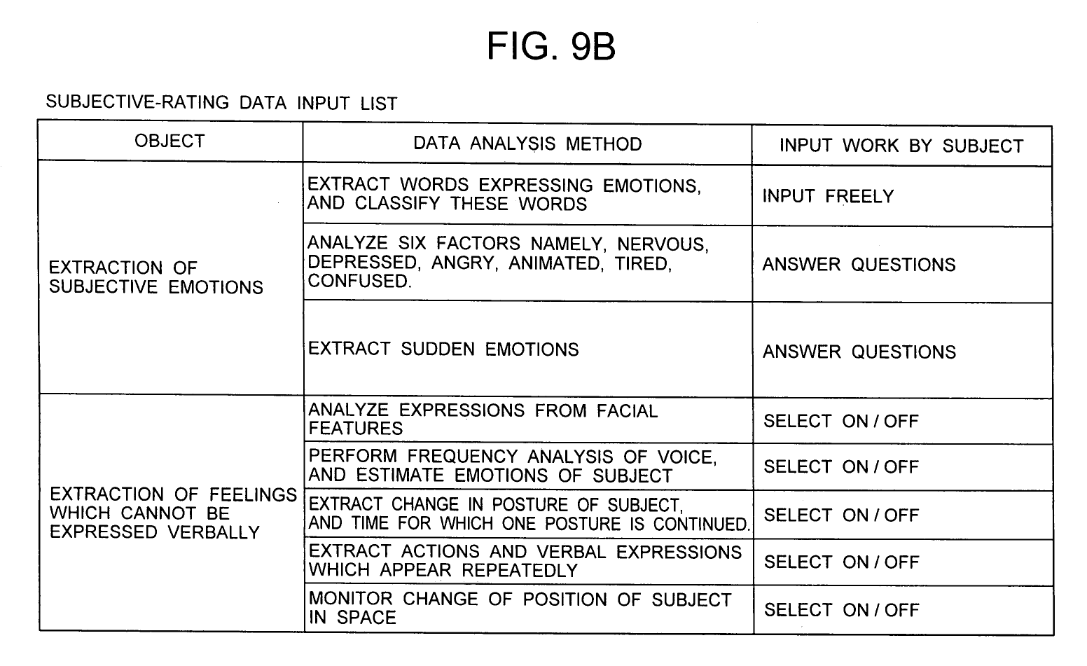

FIG. 9A and FIG. 9B show content related to subjective rating data in further detail. FIG. 9A and FIG. 9B, as a typical example of the subjective rating data, show each item example, data collection method, data analysis method, and input work by the subject, for extraction of subjective emotions and extraction of feelings which are not expressed in words.

For example, based on FIG. 9A and FIG. 9B, an example in which the subjective emotions are extracted by using an introspective method will be described as a typical example. The subject 10 creates a diary by using a key board. The control and calculating section 105, based on character data of the diary which is input, extracts words expressing the emotions of the subject 10, and perceives the events and emotions of the subject 10. The subject 10 can perform the input work freely (voluntarily).

Moreover, regarding an extraction of feelings which are not expressed in words, for example, the camera input section 112 performs a video shooting of “expressions” of the subject 10. Next, based on data which is shot, the control and calculating section 105 extracts and analyzes facial features of the subject 10.

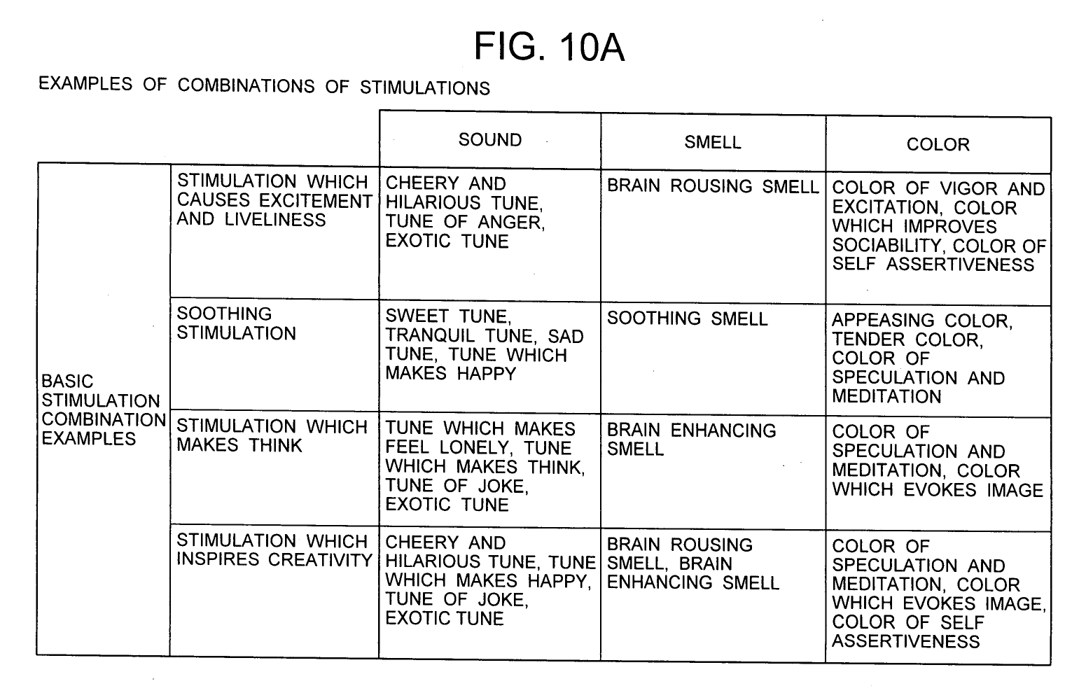

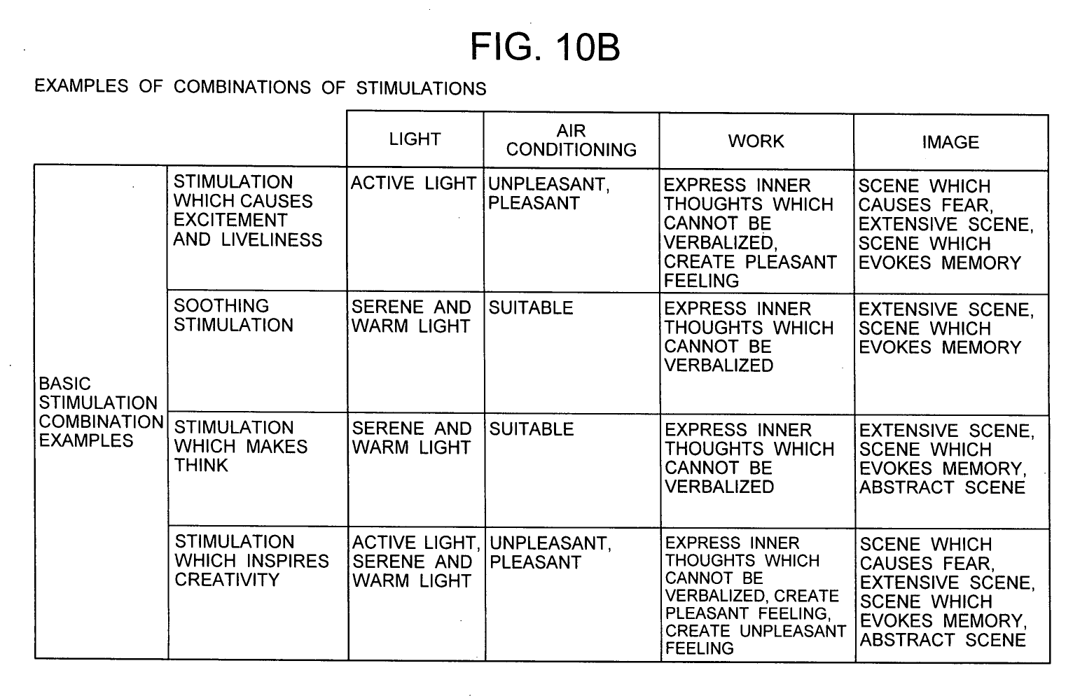

FIG. 10A and FIG. 10B show examples of combinations of stimulations. Various types of data related to “Sound”, “smell”, “color”, “light”, “air conditioning”, “work”, and “image” are stored in the database 108. For examples, the description will be made by considering a case of imparting to the subject 10 a “stimulation which causes excitement and liveliness” as a representative example. When the control and calculating section 105 has to select the “stimulation which causes excitement and liveliness”, the control and calculating section 105 selects one of a cheery and hilarious tune, a tune of anger, and an exotic tune, as “sound”, from the data stored in the database 108. The control and calculating section 105 selects a brain rousing smell as “smell”. Regarding “color”, one of a color of vigor and excitation, a color which improves sociability, and a color of self assertiveness is selected. Regarding “light”, an active light is selected. Regarding “air conditioning”, conditions of temperature and humidity, for feeling one of unpleasant condition or pleasant condition is selected. Regarding “work”, one of work of expressing inner thoughts which cannot be verbalized, and work of creating pleasant feeling is performed by the subject 10. Regarding “image”, one of a scene which causes fear, an extensive scene, and a scene which evokes memory is selected. Stimulation which is not selected here is selected at step S708.

As “image”, the video which is shot may be displayed or peculiarity for each item may be extracted and displayed. Thus, steps S702 and S705 correspond to a step of providing stimulation at which a stimulation which induces an action by the subject 10 is provided. The step S703 corresponds to a step of collecting the physiological response data, a step of collecting the subjective rating data, and a step of collecting the action rating data.

At step S705, the control and calculating section 105 selects a stimulation according to a state of mind of the subject 10, based on the physiological response data from the physiological response data collecting section 102, and the subjective rating data and the action rating data from the subjective rating-action rating data collecting section 103. The stimulation providing section 101 imparts the selected stimulation to the subject 10. The subject, by its own, can select an execution of step S702 and an execution of step S703.

At step S705, the control and calculating-section 105 based on the stimulation and any one of the physiological response data, the subjective rating data, and the action rating data, evaluates a mind-body state of the subject 10. Next, the subject 10 makes a judgment of whether or not the subject felt that a state of mind is changed.

Here, the data storage section 106 stores the physiological response data, the subjective rating data, and the action rating data which are acquired, the stimulation, and an evaluation result of the mind-body state. As the data storage section, for example, a memory can be used.

When the judgment result at step S706 is positive, at step S710, the display section 107 display the physiological response data, the subjective rating data, and the action rating data of the subject 10 corresponding to the stimulation due to which the state of mind is changed. Next, at step S711, a response of the subject 10 for the physiological response data, the subjective rating data, the action rating data, an amount of stimulation, and the stimulation at that time is output. Examples of the output are an output of the data sheet by the data sheet discharging section 143 described above, and an output to a recording medium by the USB connecting section 144. Steps S710 and S711 correspond to a step of feed back.

On the other hand, when the judgment result at step S706 is negative, at step S707, the subject 10 inputs own preference and current feeling via the camera input section 112, the key board, and the microphone.

At step S708, the control and calculating section 105 changes the content of the stimulation. Next, at step S709, the stimulating providing section 101 imparts to the subject 10 the content of stimulation which is changed. “To change the content of the stimulation” means to increase the amount of stimulation, and to vary the type of stimulation. Next, the process is returned to step S706.

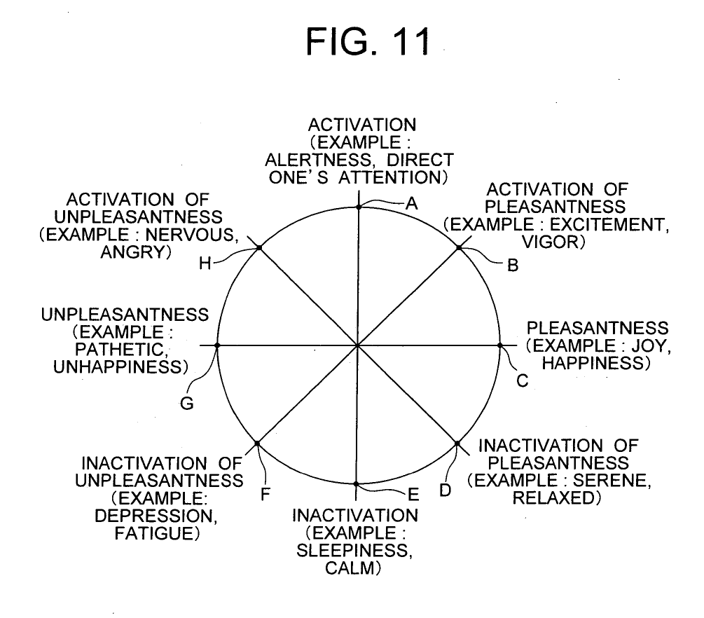

Next, a change in the state of mind of the subject 10 will be described below. FIG. 11 is a diagram for describing a method for selecting a stimulation to be provided to the subject 10, based on a “principle of homogeneity” of Altshuller which is supposed to be a basic principle of the music therapy.

Emotions of the subject 10 are let to correspond to eight positions on a circular ring as shown in FIG. 11, and are set to the following A to H. Such a circular ring diagram is called as a circumplex model of emotion (8-division mode1) by Larsen and Diener.
A . . . Activation
B . . . Activation of pleasantness
C . . . Pleasantness
D . . . Inactivation of pleasantness
E . . . Inactivation
F . . . Inactivation of unpleasantness
G . . . Unpleasantness
H . . . Activation of Unpleasantness

The stimulation is imparted to the subject 10 so as to aim at a pleasant emotion from the current emotion of the subject 10, via an adjacent emotion on the circular ring. Moreover, when the current emotion of the subject is already the pleasant emotion, the stimulation is imparted to aim at another pleasant emotion.

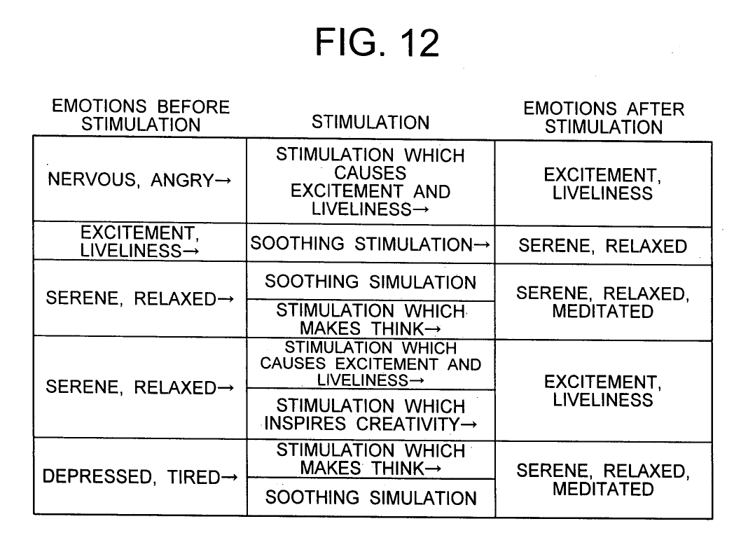

FIG. 12 shows a relationship between the stimulations and the emotions. For example, let an emotion before receiving the stimulation be a “nervous, angry (activation of unpleasantness)” state. At this time, “a stimulation which causes excitement and liveliness” is imparted to the subject 10. Accordingly, an emotion of the subject 10 after receiving the stimulation is changed to “excitement, liveliness (activation of pleasantness)”.

Next, the evaluation will be described based on three evaluation examples

First Evaluation Example:

This is an evaluation in which attention is focused on an accord and a disaccord of an externalized expressive action such as a facial expression, vocalization, and a gesture, and internalized response such as a physiological response. They are classified in the following four ways.
(1) Expressive action tendency is high, and activation of sympathetic nervous system is low→externalizer
(2) Expressive action tendency is high, and activation of sympathetic nervous system is high→generalizer—high
(3) Expressive action tendency is low, and activation of sympathetic nervous system is low→generalizer—low
(4) Expressive action tendency is low, and activation of sympathetic nervous system is high→internalizer

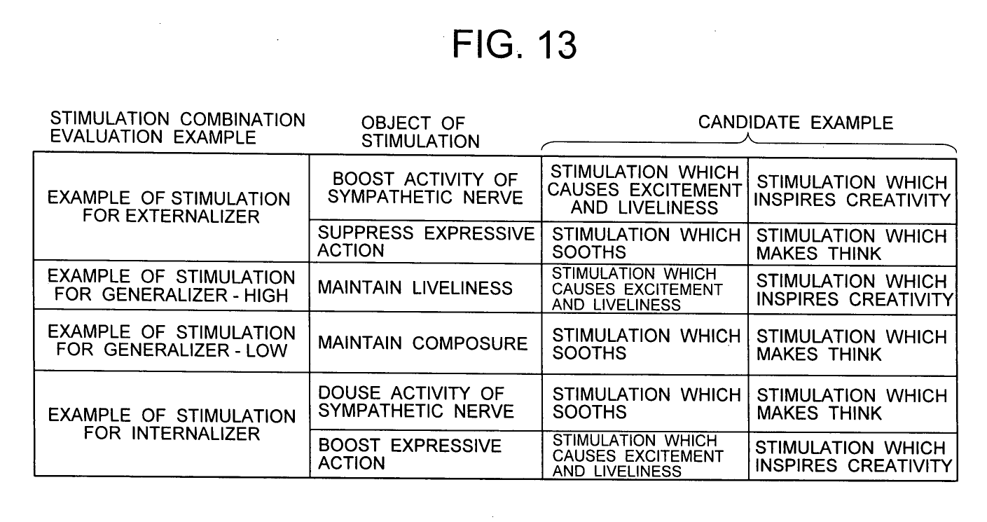

FIG. 13 shows a way of imparting stimulation in the first evaluation example. The stimulation is imparted such that the externalizer and the internalizer become generalizer. Moreover, for the generalizer—high and the generalizer—low, a stimulation which is attuned to that state is imparted.

Second Evaluation Example:

In a second evaluation example, from a verbal expression of emotion, that emotion is located on the circular ring model of the emotions described above. Examples of verbal expression of emotion are the introspective method, a POMS feeling profile test, and giving points to a plurality of basic emotions.

Third Evaluation Example:

A target object which is an integrated object derived from the verbal expression of emotion, and a degree of excitement obtained from the physiological response is located on the circular ring model of emotion described above. Examples of the verbal expression of emotion are the introspective method, the POMS feeling profile test, and giving points to the plurality of basic emotions.

Thus, in the first embodiment, by using the mind-body correlation data evaluation apparatus 100, at the step of providing stimulation, the stimulation of the content which has been set in advance is provided, or the stimulation according to the mind-body state of the subject based on one of the physiological response data, the subjective rating data, and the action rating data of the subject 10 in a state before evaluating the mind-body state is provided. Further, the content of the stimulation is changed according to the mind-body condition of the subject 10 after the stimulation is provided, and the stimulation with the changed content is provided to the subject 10. Accordingly,the subject can experience a stimulation (environment) which is suitable for the subject 10 for changing the mind-body state.

Concretely, the subject 10 can find out an effect of a chamber environment in which the subject 10 is, such as a brightness of light, the temperature, the humidity, and the sound on the physiological response, the action, and the feelings. Moreover, it is possible to detect the physiological response and action, and the feelings in various situations of the subject 10, and to impart this information to the subject 10.

For example, a question to diagnose a character trait is provided, and the subject 10 records an answer to that question. From this answer, the character trait of the subject is revealed. In addition to this, the mind-body correlation data evaluation apparatus 100 measures the physiological response, the action, and the subjectivity. Therefore, it is possible to capture a character trait which might have been hidden by the subject individual while answering, and to inform it to the subject 10.

Moreover, it is also possible to provide a question which causes make use of various senses such as a sense of sight, a sense of hearing, a sense of smell, a sense of touch, and a sense of taste. Further, the subject 10 is asked to input an answer to such stimulation by using the key board for example. The control and calculating section 105 analyses the answer of the subject 10. The data feed-back section 104 feeds back an analysis result to the subject 10. Accordingly, it is possible to make the subject 10 aware of an ability of sensory to the subject 10.

Furthermore, during the evaluation, the physiological response data, the action rating data, and the subjective rating data are recorded. Therefore, these data are analyzed and fed back to the subject 10. Accordingly, the subject 10 can be made aware of a sense which makes the subject 10 nervous, and a sense which makes the subject 10 relaxed.

Moreover, the stimulation providing section 101 can also provide a scene of having a communication with a person. The physiological response data collecting section 102 and the subjective rating—action rating data collecting section 103 detect a response to an action taken by the subject 10 at the time of stimulation. The control and calculating section 105 analyzes the data which is acquired. The data feed-back section 104 feeds back the evaluation data to the subject 10. Accordingly, it is possible to inform to the subject a tendency of an action in a scene with the other person. Furthermore, when all the data such as the physiological response data, the action rating data, and the subjective rating data during the execution of evaluation is referred to together, it is possible to inform to the subject 10 conditions of communication with a person which the subject 10 is not good at, and conditions of communication with a person the subject can be relaxed.

### SECOND EMBODIMENT

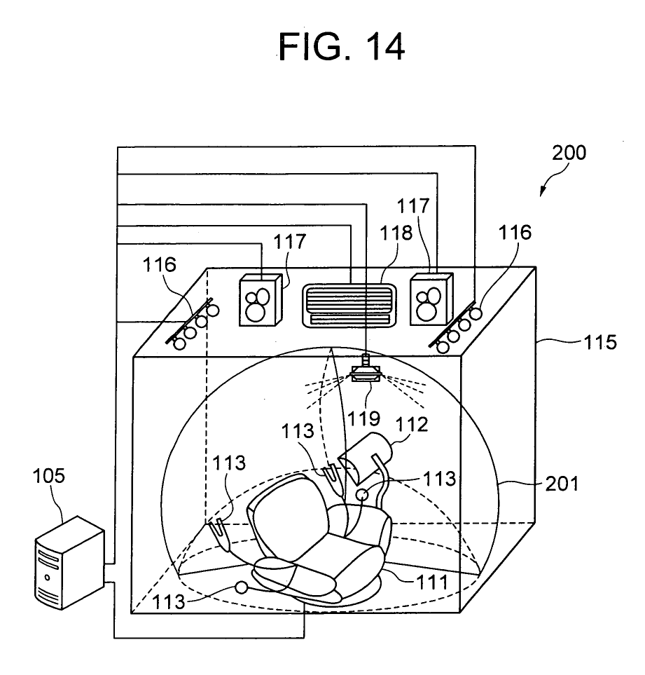

Next, a mind-body correlation data evaluation apparatus 200 according to a second embodiment of the present invention will be described below. Same reference numerals are used for components which are same as in the first embodiment, and the description which is to be repeated is omitted. FIG. 14 shows an internal structure of the mind-body correlation data evaluation apparatus 200.

In the second embodiment, an inner wall 201 has a dome shape. Therefore, the subject 10 can have a stronger immersive feeling while observing a footage etc. which is projected on the inner wall 201.

Next, a method of evaluating mind-body correlation data in the second embodiment will be described below. In the second embodiment, the mind-body correlation data evaluation apparatus 200 having the same structure as the mind-body correlation data evaluation apparatus in the first embodiment is used. Moreover, the second embodiment differs from the first embodiment at a point that in the second embodiment, a process for changing the mind-body state of the subject 10 can be selected spontaneously (voluntarily). The same reference numerals are used for the components which are same as in the first embodiment, and the description which is to be repeated is omitted.

FIG. 15 is a flowchart showing a procedure for a method of evaluating mind-body correlation data in the second embodiment. At step S1401, the subject 10 enters the mind-body correlation data evaluation apparatus 200. Here, the subject 10 is already let to have had the mind-body state evaluated by the mind-body correlation data evaluation apparatus 200 in the past. Moreover, the database 108 stores mind-body correlation data of the subject 10 which has already been acquired.

At step S1402, the mind-body correlation data of the subject 10 who has entered the chamber is acquired by the physiological response data collecting section 102, and the subjective rating-action rating collecting section 103. Moreover, the control and calculating section 105 refers to the data in the database 108 which has already been stored, and evaluates the mind-body state of the subject 10 who has entered the chamber.

At step S1403, the control and calculating section 105 refers to the data in the database 108 which has already been stored at step S1404, and provides a stimulation group according to the mind-body state of the subject 10. The providing of the stimulation group is displayed on a monitor (not shown in the diagram) equipped in the camera input section 112 for example. In the database 108, mind-body correlation data is stored at a step of storing data, at which evaluation data related to the subject which is evaluated and calculated at a step of evaluating and calculating is stored.

In the database 108, the stimulation data, the physiological response data, the subjective rating data, the action rating data related to the subject 10 are stored (accumulated) in an order of elapsing of time, for each individual. It is also possible that only the same person (subject 10) can store own data in a recording medium of the individual.

At step S1405, the subject 10, based on on experience in the past, selects an appropriate stimulation from among the stimulations which are provided. The stimulation providing section 101 provides the stimulation which is selected, to the subject 10. The step S1405 corresponds to a step of selecting stimulation.

At step S1406, a judgment of whether or not the mind-body state of the subject 10 is changed is made. When a judgment result at step S1406 is positive, at step S1407, a judgment of whether or not (the process is) to be terminated is made.

When a judgment result at step S1407 is positive, at step S1408, the display section 107 displays the physiological response data, the subjective rating data, the action rating data, the stimulation, and the response of the subject 10 to the stimulation, when the state of the mind is judged to be changed. Next, at step S1409, the physiological response data, the subjective rating data, the action rating data, the stimulation, and the response of the subject 10 to the stimulation are output. Example of the output are the output of the data sheet by the data sheet discharging section 143 described above, and an output to a recording medium by the U.S. connecting section 144. Steps S1408 and S1409 correspond to the step of feed back.

On the other hand, when the judgment result at step S1407 is negative, at step S1411, the control and calculating section 105 selects another stimulation group. Next, the stimulation group which is selected is displayed on a monitor (not shown in the diagram), which is equipped in the camera input section 112 for example. Further, step S1405 is repeated.

Moreover, when the judgment result at step S1406 is negative, the process is advanced to step S1410. At step S1410, a judgment of whether or not still another stimulation is necessary is made.

When a judgment result at step S1410 is positive, at step S1411, the control and calculating section 105 selects another stimulation group. Next, the stimulation group which is selected is displayed on the monitor (not shown in the diagram) which is equipped in the camera input section 112 for example. Further, step S1405 is repeated.

On the other hand, when the judgment result at step S1410 is negative, step S1405 is repeated till a judgment that the state of mind of the subject 10 is changed is made.

Moreover, in the second embodiment, it is desirable to perform the step of providing stimulation and the step of evaluating and calculating, at an interval. For example, the subject 10 is evaluated upon entering the mind-body correlation data evaluation apparatus 200. The data storage section 106 stores the evaluation data at this time. Moreover, the subject 10 once again enters the mind-body correlation data evaluation apparatus 200 on other day (for example, after a fixed number of days). Further, similarly, the data storage section 106 stores the evaluation data.

By repeating such procedure, the evaluation data for a plurality of evaluation dates is stored in the data storage section 106. By repeating this, the subject can make a judgment of whether the evaluation every time is heading in a satisfactory direction.

### THIRD EMBODIMENT

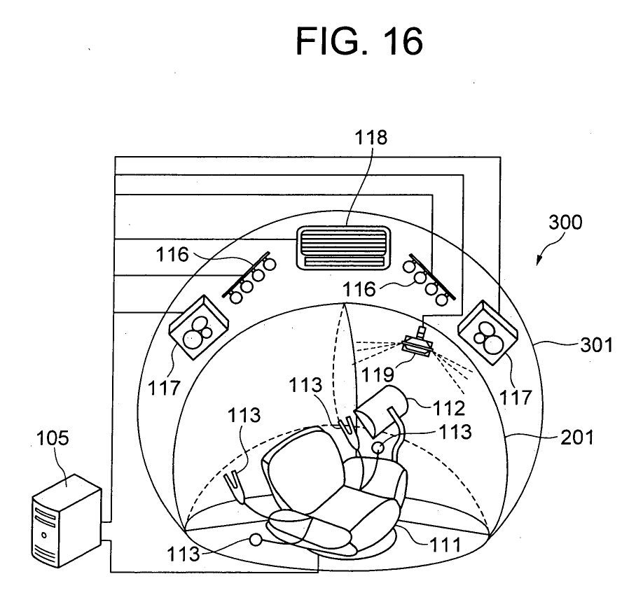

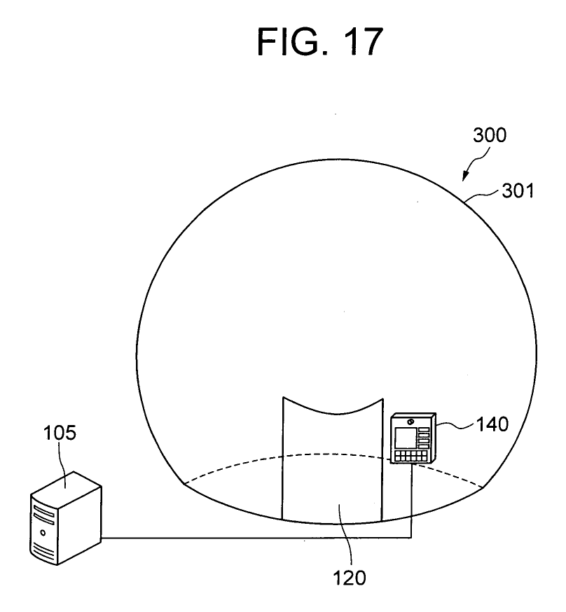

Next, a mind-body correlation data evaluation apparatus 300 according to a third embodiment of the present invention will be described below. Same reference numerals are used for components which are same as in the first embodiment, and the description which is to be repeated is omitted. FIG. 16 shows an internal structure of the mind-body correlation data evaluation apparatus 300. FIG. 17 shows an external structure of the mind-body correlation data evaluation apparatus 300.

In the third embodiment, both of a housing 301 and the inner wall 201 have the dome shape. Therefore, the subject 10 can have even stronger immersive feeling.

In the first embodiment and the second embodiment described above, an outline of the mind-body correlation data evaluation apparatus is not restricted to an apparatus having a shape of a room. For example, it is also possible to install functions of the mind-body correlation data evaluation apparatus, in a vehicle such as a car. Further, it is also possible to realize the functions of the mind-body correlation data evaluation apparatus by a wearable unit by the subject 10, and to install in a toy for children.

It is desirable to select the stimulation appropriately by classifying the content of stimulation according to a mental situation and a physical situation of the subject. For example, cases are divided into cases for both mentally and physically fit person, a mentally handicapped person, a physically handicapped person, and both mentally and physically handicapped person. Moreover, a stimulation suitable for each case is to be selected.

Further, a method for giving instructions such as selecting stimulation by the subject can be let to be a method of operating an operating section by fingers, a method of operating by audio, a method of giving a sign by breathing or by eyes, and a method of various body expressions and enlarged body expressions.

Moreover, it is desirable that the stimulation is provided such that the subject feels natural. Examples of stimulation which is felt natural are shown below.
(1) Questions are asked to the subject 10 by the mind-body correlation data evaluation apparatus in a mother language of the subject 10.
(2) Instructions are made by the subject 10 to the mind-body correlation data evaluation apparatus in the mother language of the subject 10.
(3) A communication between the subject 10 and the mind-body correlation data evaluation apparatus is let to progress like a conversation.
(4) A temperature range of the mind-body correlation data evaluation apparatus is changed such that a room temperature of the mind-body correlation data evaluation apparatus is matched with a growing ecosystem of the subject 10.
(5) For example, as when an image of sea is provided, with the image, a smell of the sea, sound of the sea, air temperature, and flow of air at the sea are also provided, other stimulation is to be provided integrally.

Moreover, the present invention is also applicable to fields such as the following. Information related to the stimulation and evaluation which are acquired without specifying the subject can be fed back to various industrial fields such as architectural designing and interior designing. Furthermore, the present invention is also applicable to a field of counseling. According to the present invention, it is possible to acquire objective data, instead of depending on subjective data of a counselor as it has been hitherto done. Thus, the present invention can take various modified embodiments which fall within the basic teaching herein set forth.

Thus, the mind-body correlation data evaluation apparatus according to the present invention is suitable as an apparatus which supports changing the mind-body state of the subject.

According to the present invention, it is possible to. provide a mind-body correlation data evaluation apparatus and a method of estimating mind-body correlation data by providing various stimulations to the subject, and by changing the mind-body state of the subject by evaluating and feeding back the response to the stimulation.

1. Mind-body correlation data evaluation apparatus comprising:
a stimulation providing section which provides stimulation to motivate an action of a subject;
a physiological response data collecting section which collects physiological response data of the subject;
a subjective rating data collecting section which collects subjective rating data of the subject;
an action rating data collecting section which collects action rating data of the subject;
an evaluating and calculating section which evaluates a mind-body state of the subject based on the stimulation, and at least one of the physiological response data, the subjective rating data, and the action rating data; and
a feed-back section which feeds back to the subject at least the mind-body state of the subject.

2. The mind-body correlation data evaluation apparatus according to claim 1, further comprising:
a data storage section which stores at least one of the physiological response data, the subjective rating data, and the action rating data; and
a database section for referring to, when the stimulation providing section provides the stimulation.

3. The mind-body correlation data evaluation apparatus according to claim 1, wherein:
the stimulation providing section includes at least one of an audio output unit, an illuminating unit, an air-conditioning unit, and an image display unit, and
the physiological response data collecting section includes probes for measuring a physiological response of the subject.

4. The mind-body correlation data evaluation apparatus according to claim 3, further comprising:
a housing in which the subject can enter or exit, which is provided with:
at least one of the audio output unit, the illuminating unit, the air-conditioning unit, and the image display unit,
the probes which measure the physiological response of the subject,
an input unit to which the subjective rating data is inputted,
a image capture instrument unit which is included in the action rating data collecting section, and
an operating section which operates the stimulation providing section.

5. The mind-body correlation data evaluation apparatus according to claim 4, wherein:
the housing includes an inner wall surface having a dome shape, and
at least the image display unit is provided on an inner side of the inner wall surface.

6. Method of evaluating mind-body correlation data comprising the steps of:
providing stimulation at which, a stimulation which motivates an action of a subject is provided;
collecting physiological response data at which, physiological response data of the subject is collected;
collecting subjective rating data at which, subjective rating data of the subject is collected;
collecting action rating data at which, action rating data of the subject is collected;
evaluating and calculating at which, a mind-body state of the subject is evaluated based on the stimulation, and at least one of the physiological response data, the subjective rating data, and the action rating data; and
feeding back at which, at least the mind-body state of the subject is fed back to the subject.

7. The method of evaluating mind-body correlation data according to claim 6, wherein:
at the step of providing stimulation, a stimulation which is determined in advance is provided, or a stimulation according to the mind-body state of the subject based on at least one of the physiological response data, the subjectivity rating data, and the action rating data of the subject in a state before evaluation of the mind-body state, is provided, and
a stimulation is provided to the subject after changing content of stimulation according to the mind-body state of the subject after the stimulation is provided.

8. The method of evaluating mind-body correlation data according to claim 6, further comprising the steps of:
storing data at which at least evaluation data related to the subject from the step of evaluating and calculating is stored; and
selecting stimulation at which the subject is made to select a stimulation from candidates for stimulation which are provided, and wherein:
at the step of providing stimulation, the candidates for stimulation according to the mind-body state of the subject are provided based on at least the evaluation data stored at the step of storing data.

9. The method of evaluating mind-body correlation data according to claim 6, wherein:
the step of providing stimulation and the step of evaluating and calculating are performed at an interval of time.

10. The method of evaluating mind-body correlation data according to claim 7, wherein:
the step of providing stimulation and the step of evaluating and calculating are performed at an interval of time.

11. The method of evaluating mind-body correlation data according to claim 8, wherein:
the step of providing stimulation and the step of evaluating and calculating are performed at an interval of time.
